(19) United States
(12) Patent Application Publication
(10) Pub. No.: US 2014/0248370 A1
(43) Pub. Date: Sep. 4, 2014

(54) Enhancement Composition

(71) Applicant: Abdulkadir Hashi Ali, Columbus, OH (US)

(72) Inventor: Abdulkadir Hashi Ali, Columbus, OH (US)

(21) Appl. No.: 14/197,052

(22) Filed: Mar. 4, 2014

Related U.S. Application Data
(60) Provisional application No. 61/772,512, filed on Mar. 4, 2013.

(51) Int. Cl. A61K 36/328; A61K 36/71; A61K 36/88; A61K 35/20; A61K 31/20; A61K 8/36; A61K 31/202; A61K 31/201; A61K 36/185; A61K 8/92; A61K 8/97; A61K 36/324; A61K 36/258

(52) U.S. Cl. CPC A61K 36/328; A61K 36/324; A61K 36/71; A61K 36/88; A61K 35/20; A61K 31/20; A61K 36/258; A61K 31/202; A61K 31/201; A61K 36/185; A61K 8/922; A61K 8/925; A61K 8/97; A61K 8/361
USPC 424/535; 424/195.18

(57) ABSTRACT

An enhancement composition includes a combination of natural ingredients that form a synergy to enhance the health, appearance, smell, and psyche of a surface, such as skin. Unnatural or processed ingredients are not included that can cause irritation and more detrimental effects to the skin. The enhancement composition has no preservatives because the particular combination of elements in the cream disclosed herein preserves the cream and permits a long shelf life. The enhancement composition is fabricated by combining, mixing, and sometimes heating predetermined amounts of Frankincense, Myrrh, black seed, and aloe. Additional ingredients can include, Shea butter, camel hump butter, coconut oil, olive oil, ginseng, natural resins, fatty acids, and various oils. The ingredients are combined by mixing, heating, cooling, distilling, or applying radiation. The composition promotes of spiritual health, particularly when implemented to include Frankincense and Myrrh.

### CROSS-REFERENCE TO RELATED APPLICATIONS

The present Utility patent application claims priority benefit of the U.S. provisional application for patent Ser. No. 61772512 entitled “Enhancement Composition”, filed on 4, Mar. 2013, under 35 U.S.C. 119(e). The contents of this related provisional application are incorporated herein by reference for all purposes to the extent that such subject matter is not inconsistent herewith or limiting hereof.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

One or more embodiments of the invention generally relate to an enhancement composition. More particularly, the invention relates to an enhancement composition that creates synergy between a plurality of natural ingredients to provide a physically enhancing composition.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that cream is a topical preparation usually for application to the skin. Creams and lotions used in the cosmetic industry consist of semi-solid emulsions, and formed from a mixture of oils and water.

Typically, topical creams and lotions applied to various external parts of the human body, such as the skin and hair. The creams are efficacious for cleaning, perfuming, or protecting, to keep the skin or hair in good condition, change the appearance, or correct body odors. In many instances, the cram can act as a barrier to protect the skin. The topical cream can also be used to clean the skin and contain one or more drug substances dissolved or dispersed in a suitable base. The topical cream can have both natural and synthetic ingredients.

In many instances, topical lotions and creams can be used for medicinal, cosmetic, and therapeutic reasons. The lotions often contain essential oils. The essential oils Essential oils are often used alone or in combination to provide therapeutic healing benefits when applied to the skin and to provide a fragrance that is both appealing and beneficial.

It is known that Frankincense is an aromatic resin obtained from trees of the genus Boswellia. Myrrh is the aromatic resin of a number of small, thorny tree species of the genus Commiphora. Both Frankincense and Myrrh have cosmetic, medical, and spiritual benefits.

Frankincense is now identified as a one of a kind treatment for conditions such as asthma, rheumatoid arthritis and atopic dermatitis. Studies also have also found anti-cancer properties along with the anti-inflammatory properties.

Nature's answer for wound repair

The powder of the dried resin is a common ingredient of herbal pastes to treat wounds. A study from the Hebrew University in Israel, indicated an isolate from frankincense resin reduces neurological damage from injuries.

The resin has been used to support oral health for thousands of years in Middle Eastern areas. It is used to treat infections of the teeth and gums. This is understandable since frankincense oil is a good anti-septic and disinfectant; it eliminates germs just from the fumes alone. It can also be applied on wounds to protect them from tetanus and infection. To elevate your mood a few drops—as a perfume—works well. Frankincense is used in skin care products and is known for its ability to prevent and reduce wrinkles.

To stop itching skin apply a few drops of Frankincense to the affected area. Rub on sore joints to relieve pain from an injury or arthritis. For cuts and scrapes, apply one drop to clean and disinfect. To remove a wart apply topically to the area a couple times a day and have patience; it can take a few weeks to get the results you want.

For colds—it is used by infusing the room with the aroma; this seems to relive congestion. When it comes to using it internally—a healthcare provider can help. This is a very potent and strong oil and as such must be pure and the dosage has to be correct.

By way of example, see such known benefits at: http://www.naturalhealth365.com/natural_healing/frankincense.html#sthash.yIDWmKX Q.dpuf

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates a detailed perspective view of an exemplary enhancement composition removing residue from an exemplary surface, in accordance with an embodiment of the present invention;

FIGS. 2A and 2B illustrate perspective views of exemplary natural ingredients, in accordance with an embodiment of the present invention, where FIG. 2A illustrates exemplary Frankincense, and FIG. 2B illustrates an exemplary Myrrh tree;

FIG. 3 illustrates a flowchart diagram for an exemplary method for manufacturing an exemplary enhancement composition, in accordance with an embodiment of the present invention; and

FIGS. 4A, 4B, 4C, and 4D illustrate various formulation tables for an exemplary enhancement composition, in accordance with an embodiment of the present invention, where FIG. 4A illustrates a first formulation, FIG. 4B illustrates a second formulation, FIG. 4C illustrates a second formulation, and FIG. 4D illustrates a second formulation, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub- steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

There are various types of enhancement compositions that may be provided by preferred embodiments of the present invention. In one embodiment of the present invention, the enhancement composition may include a natural chemical composition that forms a synergy between a plurality of natural ingredients to provide a composition for enhancing the health, appearance, smell, and psyche of a user. The plurality of ingredients may be combined in various amounts and phases for producing the desired enhancement composition. Those skilled in the art will recognize that excessive and unnatural ingredients may cause irritation and more detrimental effects to the skin than if nothing was applied. Numerous cosmetics utilize caustic and man made chemicals. These types of chemicals may cause side effects that are undesirable and result in adverse side effects that outweigh the benefits.

In some embodiments, the plurality of ingredients may include natural resins, herbs, fats, and plants that serve to enhance the health, appearance, and psyche of a user. The combinative effects of the enhancement composition may allow each ingredient to function more effectively. In some embodiments, the plurality of natural ingredients may include, without limitation, Frankincense, Myrrh, aloe, black seed, Shea butter, camel hump fat, coconut oil, olive oil, and ginseng, fatty acids, and oils. In some embodiments, the plurality of ingredients may be combined by standard laboratory procedure, including, without limitation, mixing, heating, cooling, distilling, and applying radiation. Since the ingredients are all natural, and generally nonvolatile, myriad combinations and percent compositions may be utilized during manufacturing to achieve the desired results. The enhancement composition may be made by mixing and heating predetermined amounts of the natural ingredients in various quantities.

The enhancement composition may be used for sensitive skin, including skins that are allergic to components of commercially-available creams and topical ointments. In its purest form, the enhancement composition has no preservatives and needs none because the particular combination of elements in the cream disclosed herein preserves the cream and permits a long shelf life. Additionally, the enhancement composition utilizes a basic composition that is easy to manufacture. Yet another benefit of the enhancement composition may be the promotion of spiritual health, particularly when implemented to include Frankincense and Myrrh.

In one embodiment of the present invention, the resin used in the enhancement composition may include, without limitation, Frankincense, Myrrh, elemi, turpentine, copaiba. Those skilled in the art, in light of the present teachings, will recognize that Frankincense may be characterized by a balsamic-spicy, slightly lemon, fragrance of incense, with a conifer-like undertone. The Frankincense may include anti-inflammatory properties efficacious for the treatment of joint pain and arthritis. In some embodiments, the Frankincense oil may be utilized for treating medical maladies, including, without limitation, rejuvenating the skin, and treating acne, bacterial, and fungal infections, wounds, and scars. Frankincense may also be efficacious for enhancing aging and dry skin. The Frankincense may further provide an elevating, soothing quality that calms the nervous system. In one embodiment, the composition of Frankincense may include, without limitation, about 56% acid resin, soluble in alcohol and having the formula: C₂₀H₃₂O₄, gum, similar to gum arabic 30-36%; 3-acetyl-beta-boswellic acid; alpha-boswellic acid; 4-O-methyl-glucuronic acid; incensole acetate; and phellandrene.

In one embodiment of the present invention, the Myrrh may include a reddish resin originating from species of the genus Commiphora. The Myrrh may be utilized for treating medical maladies, including, without limitation, appetite regulation, anger, asthma, athlete's foot, bedsores, boils, bronchitis, chapped skin, colds, coughs, digestive system, eczema, gingivitis, hemorrhoids, mouth ulcers, rejuvenate mature complexions, rheumatism, skin ulcers, sore throats, sores, spongy gums, stress, viral infections, wounds, and wrinkles. In some embodiments, the Shea butter may include an ivory-colored fat extracted from the nut of an African Shea tree, and utilized as a balm. Shea butter may be comprised of a triglyceride derived mainly from stearic acid and oleic acid. The Shea butter may include a plurality of fatty acids, including, without limitation, about 40-60% oleic acid, about 20-50% stearic acid, about 3-11% linoleic acid, about 2-9% palmitic acid, about less than 1% linolenic acid, and about less than 1% arachidic acid. The Shea butter may be utilized in cosmetics as a moisturizer, salve, or lotion. In some embodiments, the Shea butter may be edible, used in food preparation, and/or serve as a prophylactic.

In yet another embodiment, the camel butter may include numerous nutrients that benefit the digestive tract and the skin, including, without limitation, vitamins, minerals, proteins, and immunoglobulins. Those skilled in the art, in light of the present teachings, will recognize that camel milk cannot be made into butter by the traditional churning method. The camel milk may be churned into camel butter if soured first, churned, and a clarifying agent added. Consequently, at least one of the ingredients in the enhancement composition may require formulation. The camel may further contribute to the ingredients by providing camel hump fat from the hump, which simply comprises of fatty tissue. In some embodiments, the black seed may be derived from an annual flowering plant, Nigella sativa. The black seed may include a fragrance. However, in other embodiments, the black seed may be consumed as a spice. In some embodiments, the aloe may originate from a variety of flowering succulent plants. The aloe may include, without limitation, Aloe vera, efficacious for enhancing digestion, moisturizing skin, and providing a fragrance. Frankincense and Myrrh added to natural skin brightening and anti aging such as Alpha Arbutin, a skin lightening cream, is used to reduce dark spots. This cream can be used on your body as well. Alpha Arbutin (see known profile at http://www.ehow.com/facts_{—}5728822_information-alpha- arbutin.html#ixzz2ux0RFpl) helps skin to have a more even color tone. Bearberry extract, lactic acid, and emblica powder are just some of the active ingredients. Moreover, O.D.A.white™ is known to to treat skin pigmentation disorders such as freckles, melasma, hyperpigmentation and general dark colour of the ethnic skin. And MELASLOW ph for dark spot solution. It is contemplated by an embodiment of the present invention that these three ingredients when combined with Frankincense and Myrrh will provide an enhanced brightening skin cream.

In one embodiment of the present invention, the enhancement composition may combine the plurality of natural ingredients in a variety of compositions to derive health, cosmetic, and psyche enhancing compositions, including, without limitation, fragrances, incense, aromatherapy candles, soap, shampoos, shower gels, creams, lotions, body creams, lip stick, deodorants, facemasks, hair products, baby shampoos, diapers, wiping baby bottoms, anitseptic gum, candy, prophylactics, and antiviruses. In one embodiment, the enhancement composition may include a combination of Frankincense, Myrrh, black seed, and aloe for producing a face cream. In yet another embodiment, the enhancement composition may include a combination of Frankincense, Myrrh, camel hump butter, Shea butter, and aloe for producing a soap. In yet another embodiment, the enhancement composition may include a combination of Frankincense, Myrrh, Ginseng, and camel hump butter, Shea butter. In yet another embodiment, the enhancement composition may include a combination of Frankincense, Myrrh, and any natural ingredient efficacious for enhancing health, appearance, smell, and psyche.

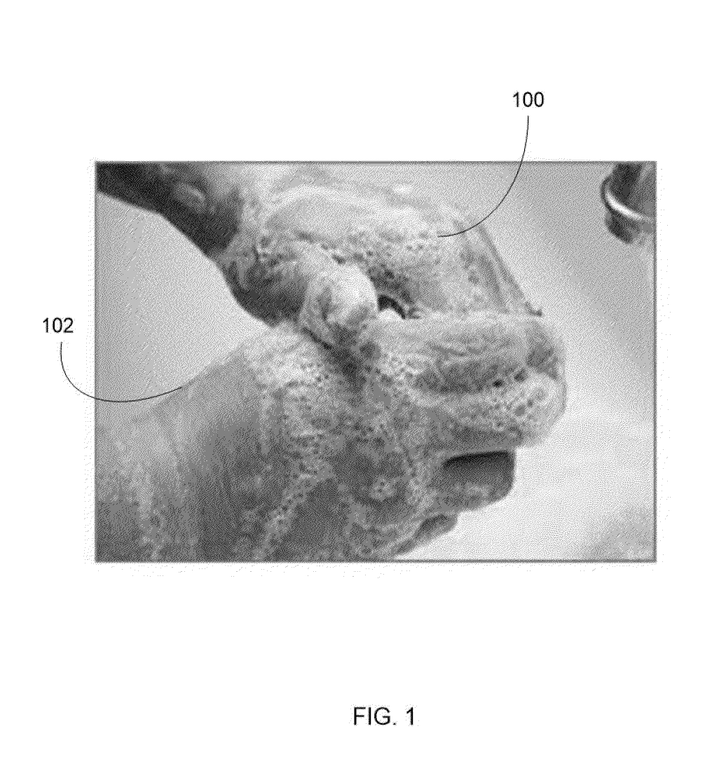

FIG. 1 illustrates a detailed perspective view of an exemplary enhancement composition removing residue from an exemplary surface, in accordance with an embodiment of the present invention. In the present invention, an enhancement composition 100 may include a combination of natural ingredients that form a synergy to enhance the health, appearance, smell, and psyche of a user. Because only natural ingredients are used, unnatural or processed ingredients are not included that may cause irritation and more detrimental effects to the skin. Numerous cosmetics utilize more in chemicals than organic herbs, causing side effects from chemicals that are undesirable and resulting in side effects that outweigh the benefits.

For example, without limitation, a Frankincense ingredient may be combined with a Shea butter ingredient, a lye ingredient, and a water ingredient to form an enhancement composition, including, a surfactant for removing residue from a surface 102. The surfactant may include, without limitation, soap having an aromatic fragrance. In some embodiments, the plurality of natural ingredients may be combined in various amounts and phases for producing the surfactant, or any variety of other enhancement compositions. In some embodiments, the plurality of natural ingredients may include natural resins, herbs, fats, and plants that serve to enhance the health, appearance, and psyche of a user.

The enhancement composition may be made by mixing and heating predetermined amounts of the natural ingredients in various quantities. The combinative effects of the enhancement composition may allow each ingredient to function more effectively. In some embodiments, the plurality of natural ingredients may include, without limitation, Frankincense, Myrrh, black seed, and aloe. Additional ingredients may also be included with these base ingredients, including without limitation, aloe, Shea butter, camel hump butter, coconut oil, olive oil, ginseng, natrual resins, fatty acids, and various oils. In some embodiments, the plurality of ingredients may be combined by standard laboratory procedure, including, without limitation, mixing, heating, cooling, distilling, and applying radiation. For example a measured quantity of each ingredient is added while slowly heating or cooling the entire composition.

In some embodiments, production of the enhancement composition may include initially mixing the Frankincense, Myrrh, black seed, often with a fragrance, and in a blender. The oils from these natural ingredients may be separately combined together with a substance, such as vitamin E, and heated at a low heat. However in other embodiments, heating is not necessitated, and the oils separate from mixing. The resulting composition may provide medicinal benefits, therapeutic benefits, cosmetic benefits, and, to some users, spiritual benefits through the inclusion of the Frankincense, Myrrh. In addition, in its purest form, without the various optional ingredients mentioned above, the enhancement composition may be hypoallergenic, thereby providing the foregoing medicinal, therapeutic, and spiritual benefits to all users, regardless of skin sensitivities.

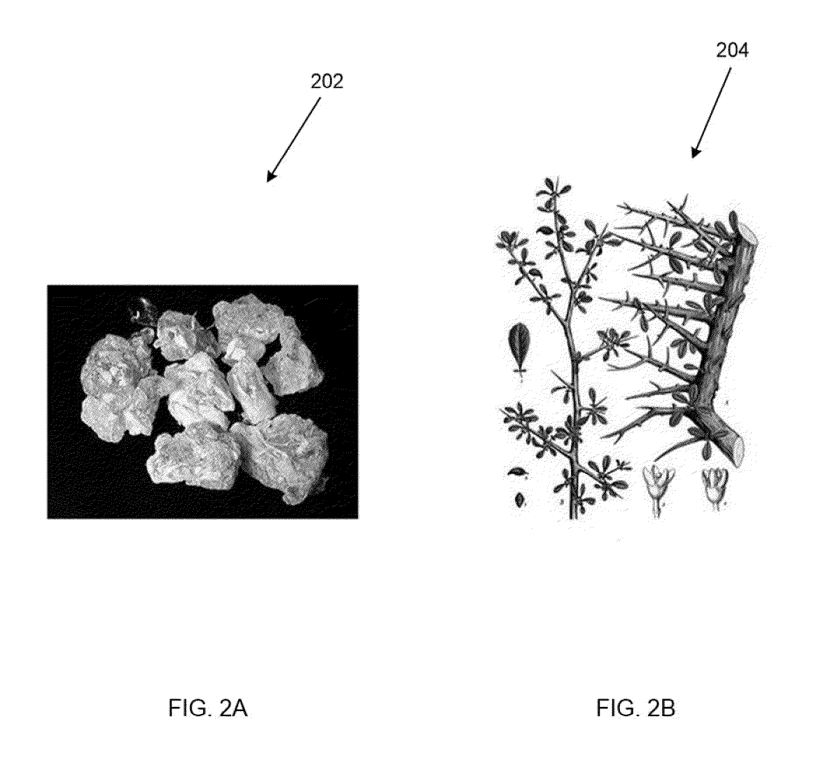

FIGS. 2A and 2B illustrate perspective views of exemplary natural ingredients, in accordance with an embodiment of the present invention, where FIG. 2A illustrates exemplary Frankincense, and FIG. 2B illustrates an exemplary Myrrh tree. In the present invention, the Frankincense 202 may be characterized by a balsamic-spicy, slightly lemon, fragrance of incense, with a conifer-like undertone. The Frankincense may include anti-inflammatory properties efficacious for the treatment of joint pain and arthritis. In some embodiemnts, the Frankincense oil may be utilized for treating medical maladies, including, without limitation, rejuvenating the skin, and treating acne, bacterial, and fungal infections, wounds, and scars. Frankincense may also be efficacious for enhancing aging and dry skin. The Frankincense may further provide an elevating, soothing quality that calms the nervous system.

In one embodiment, the composition of Frankincense may include, without limitation, about 56% acid resin, soluble in alcohol and having the formula: C₂₀H₃₂O₄, gum, similar to gum arabic 30-36%; 3-acetyl-beta-boswellic acid; alpha-boswellic acid; 4-O-methyl-glucuronic acid; incensole acetate; and phellandrene. In one embodiment, the Myrrh 204 may include a reddish resin originating from species of the genus Commiphora. The Myrrh may be utilized for treating medical maladies, including, without limitation, appetite regulation, anger, asthma, athlete's foot, bedsores, boils, bronchitis, chapped skin, colds, coughs, digestive system, eczema, gingivitis, hemorrhoids, mouth ulcers, rejuvenate mature complexions, rheumatism, skin ulcers, sore throats, sores, spongy gums, stress, viral infections, wounds, and wrinkles.

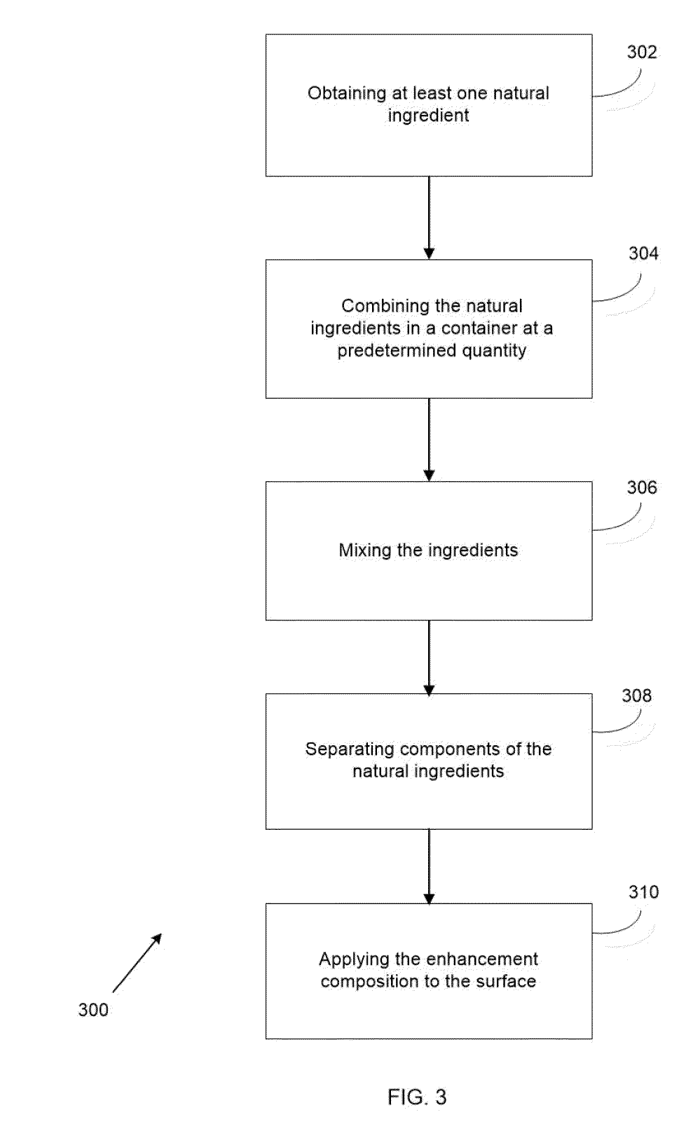

FIG. 3 illustrates an exemplary method for manufacturing an exemplary enhancement composition, in accordance with an embodiment of the present invention. In the present invention, a method 300 for manufacturing the enhancement composition may include numerous steps that combine various types of natural ingredients. In some embodiments, the method may combine the natural ingredients to form the enhancement composition. The enhancement composition may be manufactured into a variety of compositions to derive health, cosmetic, and psyche enhancing compositions, including, without limitation, fragrances, incense, aromatherapy candles, soap bar, shampoos, shower gels, creams, lotions, body creams, lip stick, deoderants, facemasks, hair products, baby shampoos, diapers, wiping baby bottoms, anitseptic gum, candy, prophylactics, and antiviruses. Other uses of the present composition include skin brightening, Anti aging creams, and dark spot cream solutions.

In some embodiments, the method may include an initial Step 302 of obtaining at least one natural ingredient. The natural ingredient may include, without limitation, the plurality of natural ingredients may include, without limitation, Frankincense, Myrrh, black seed, and aloe. Additional ingredients may also be included with these base ingredients, including without limitation, aloe, Shea butter, camel hump butter, coconut oil, olive oil, ginseng, natrual resins, fatty acids, and various oils.

A Step 304 may include combining the natural ingredients into a container in a predetermined quantity. In some embodiments, the plurality of ingredients may be combined by standard laboratory procedure, including, without limitation, mixing, heating, cooling, distilling, and applying radiation. The quantities may be varied, depending on the required uses for the enhancement composition. For example, without limitation, a skin salve may utilize additional quantities of the aloe. A spiritual use for the composition may require simply combining Frankincense and Myrrh oils. In some embodiments, a Step 306 may include mixing the ingredients. The mixing or blending may be performed with a standard blender known in the art. The mixing may help separate the oils from the natural ingredients. One example composition of the present embodiment may include, without limitation, a mixture for a skin brightening cream comprised of approximately 2% Frankincense, 1% Myrrh, 8% Alpha Arbutin, 5% Melaslow PH and 3% O.D.A. WHITE.

In some embodiments, a Step 308 may include separating components of the natural ingredients in the enhancement composition. The separation may be efficacious for removing chaffe, and other undesirable parts of the natural ingredients that provide no benefits to the enhancement composition. The separation may include, without limitation, distillation, and thereby require heating. A final Step 310 may include applying the enhancement composition to the surface. The surface may include skin. The application may include rubbing, massaging, and other topical applications. The enhancement composition may also be slightly heated to assist in application to the surface, by creating greater viscosity. In some embodiments, the enhancement composition may be efficacious for treating medical maladies, including, without limitation, rejuvenating the skin, and treating acne, bacterial, and fungal infections, wounds, and scars. The enhancement composition may also help enhance aging and dry skin. The enhancement composition may further provide an elevating, soothing quality that calms the nervous system.

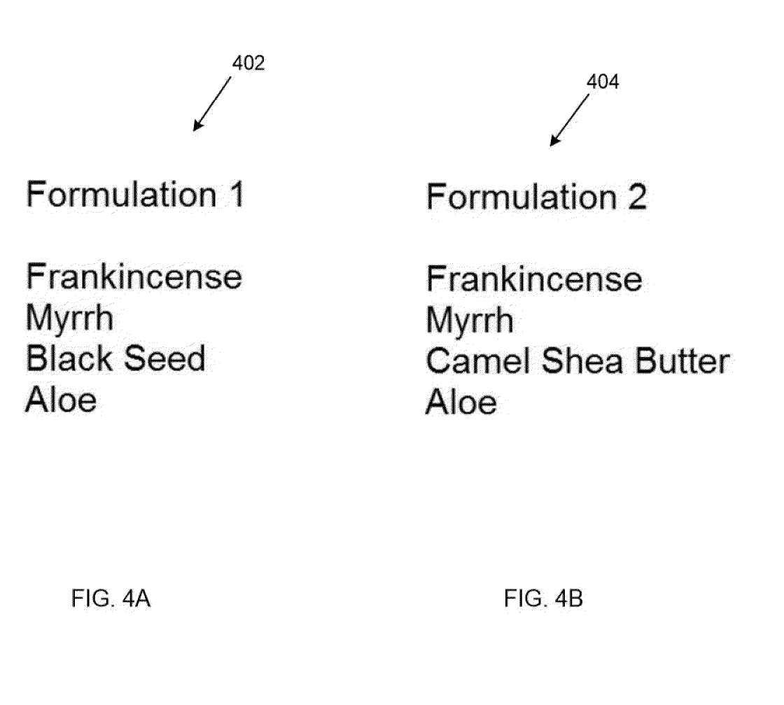

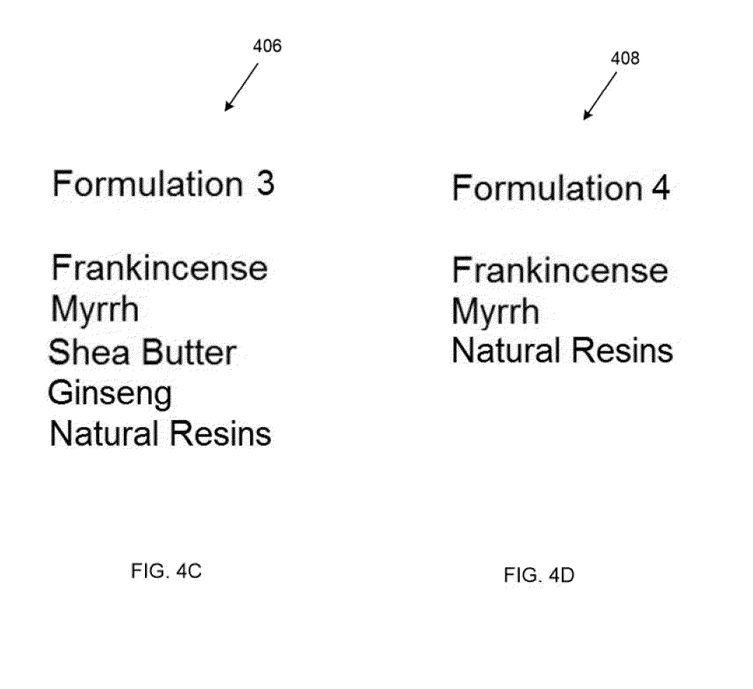

FIGS. 4A, 4B, 4C, and 4D illustrate formulation tables of an exemplary enhancement composition, in accordance with an embodiment of the present invention, where FIG. 4A illustrates a first formulation, FIG. 4B illustrates a second formulation, FIG. 4C illustrates a second formulation, and FIG. 4D illustrates a second formulation. In one embodiment, the enhancement composition may include a first formulation 402 consisting of Frankincense, Myrrh, black seed, and aloe. The first formulation may produce a soap, comprising about 25%-35% Frankincense, about 15%-30% Myrrh, about 10%-20% Aloe Vera, and about 5%-10% Black seed.

In one embodiment, the enhancement composition may include a second formulation 404 that is similar to the first composition. The second composition may include additional ingredients that provide different types of enhancements. The second composition may consist of, a combination of Frankincense, Myrrh, Shea butter, and aloe for producing a facial cream. However, other embodiments may include combinations of Frankincense, Myrrh, Ginseng, and Shea butter; or combinations of Frankincense, Myrrh, and any natural ingredient efficacious for enhancing health, appearance, smell, and psyche.

In one embodiment, the enhancement composition may include a third formulation 406 comprising Frankincense, Myrrh, Shea butter, ginseng, and natural resins. The additional of ginseng is commonly used in hair tonics and cosmetics. In some embodiemnts, a fourth formulation 408 may include simply Frankincense, Myrrh, and natural resins. However, in any of the aforementioned formulations, additional natural ingredients, fragrances, oils, and natural medicinals may be used. Skin brightening and anti aging creams of the present embodiment, without limitation, may be comprised of approximately 2% Frankincense, 1% Myrrh, 8% Alpha Arbutin, 5% Melaslow PH and 3% O.D.A. WHITE and other essential oils these are active ingredients.

In one alternative embodiment, the enhancement composition may be utilized to help heal skin diseases, such as bacterial, fungal, parasitic, and viral skin diseases. The enhancement composition may also serve as a salve for cuts and burns, whereby ingredients, such as the aloe help soothe the burn. In yet another alternative embodiment, the enhancement composition may be manipulated with thermal energy to achieve a desired color or fragrance. In yet another alternative embodiment, the enhancement composition may be incorporated into foods for improving digestion and providing aromatic qualities to the food. In one alternative embodiment, the enhancement composition may comprise ingredients efficacious for producing food products, including, without limitation, cereal, baby food, energy bars, salad dressing, and ice cream.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

It will be further apparent to those skilled in the art that at least a portion of the novel method steps and/or system components of the present invention may be practiced and/or located in location(s) possibly outside the jurisdiction of the United States of America (USA), whereby it will be accordingly readily recognized that at least a subset of the novel method steps and/or system components in the foregoing embodiments must be practiced within the jurisdiction of the USA for the benefit of an entity therein or to achieve an object of the present invention. Thus, some alternate embodiments of the present invention may be configured to comprise a smaller subset of the foregoing means for and/or steps described that the applications designer will selectively decide, depending upon the practical considerations of the particular implementation, to carry out and/or locate within the jurisdiction of the USA. For example, any of the foregoing described method steps and/or system components which may be performed remotely over a network (e.g., without limitation, a remotely located server) may be performed and/or located outside of the jurisdiction of the USA while the remaining method steps and/or system components (e.g., without limitation, a locally located client) of the forgoing embodiments are typically required to be located/performed in the USA for practical considerations. In client-server architectures, a remotely located server typically generates and transmits required information to a US based client, for use according to the teachings of the present invention. Depending upon the needs of the particular application, it will be readily apparent to those skilled in the art, in light of the teachings of the present invention, which aspects of the present invention can or should be located locally and which can or should be located remotely. Thus, for any claims construction of the following claim limitations that are construed under 35 USC §112 (6) it is intended that the corresponding means for and/or steps for carrying out the claimed function are the ones that are locally implemented within the jurisdiction of the USA, while the remaining aspect(s) performed or located remotely outside the USA are not intended to be construed under 35 USC §112 (6).

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC §112 (1), all claims must be supported by sufficient disclosure in the present patent specification, and any material known to those skilled in the art need not be explicitly disclosed. However, 35 USC §112 (6) requires that structures corresponding to functional limitations interpreted under 35 USC §112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC §112 (6) when such corresponding structures are not explicitly disclosed in the foregoing patent specification. Therefore, for any invention element(s)/structure(s) corresponding to functional claim limitation(s), in the below claims interpreted under 35 USC §112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC §112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC §112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC §112 (6). Applicant(s) note that all the identified documents above which are incorporated by reference to satisfy 35 USC §112 (6) necessarily have a filing and/or publication date prior to that of the instant application, and thus are valid prior documents to incorporated by reference in the instant application.

Having fully described at least one embodiment of the present invention, other equivalent or alternative methods of implementing an enhancement composition for the skin that combines natural ingredients according to the present invention will be apparent to those skilled in the art. Various aspects of the invention have been described above by way of illustration, and the specific embodiments disclosed are not intended to limit the invention to the particular forms disclosed. The particular implementation of the enhancement composition for the skin that combines natural ingredients may vary depending upon the particular context or application. By way of example, and not limitation, the enhancement composition for the skin that combines natural ingredients described in the foregoing were principally directed to the combination of Frankincense, Myrrh, black seed, and aloe implementations; however, similar techniques may instead be applied to medical salves to soothe burned skin, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The Abstract is provided to comply with 37 C.F.R. Section 1.72(b) requiring an abstract that will allow the reader to ascertain the nature and gist of the technical disclosure. It is submitted with the understanding that it will not be used to limit or interpret the scope or meaning of the claims. The following claims are hereby incorporated into the detailed description, with each claim standing on its own as a separate embodiment.

What is claimed is:

1. A composition comprising:
a plurality of ingredient selected from a group of ingredients consisting of Frankincense, Myrrh, black seed, and aloe.

2. The composition of claim 1, in which said composition comprises an enhancement composition.

3. The composition of claim 2, in which said composition is configured to be applied to human skin, said application comprising rubbing or massaging.

4. The composition of claim 3, in which said group of ingredients to select from further consists of Alpha Arbutin, O.D.A. White, Octadecenedioic Acid, and MELASLOW PH, and Citrus Unshiu Peel Extract.

5. The composition of claim 4, in which said composition is configured to help lubricate said skin.

6. The composition of claim 5, in which said composition is configured to help reform said skin.

7. The composition of claim 6, in which said composition comprises all natural ingredients.

8. The composition of claim 7, in which said plurality of natural ingredients is configured to be combined and mixed.

9. The composition of claim 8, in which said mixing is operable to help separate an oil from said Frankincense and said Myrrh, said oil configured to help enhance said surface.

10. The composition of claim 9, in which said composition comprises a cream.

11. The composition of claim 10, in which said Frankincense comprises C₂₀H₃₂O₄, gum, similar to gum arabic 30-36%; 3-acetyl-beta-boswellic acid; alpha-boswellic acid; 4-O-methyl-glucuronic acid; incensole acetate; and phellandrene.

12. The composition of claim 11, in which said Myrrh comprises a reddish resin, said reddish resin originating from a species of genus Commiphora.

13. The composition of claim 12, in which the plurality of ingredients further comprises camel hump butter, and/or shea butter, and/or ginseng, and/or a fatty acid, and/or a natural resin, and/or an oil.

14. The composition of claim 13, in which said composition comprises a first formulation, a second formulation, a third formulation, and a fourth formulation.

15. The composition of claim 14, in which said first formulation comprises Frankincense, Myrrh, black seed, and aloe.

16. The composition of claim 15, in which said second composition comprises Frankincense, Myrrh, camel butter, shea butter, and aloe.

17. The composition of claim 16, in which said third formulation comprises Frankincense, Myrrh, shea butter, ginseng, and a natural resin.

18. The composition of claim 17, in which said fourth formulation comprises Frankincense, Myrrh, and a natural resin.

19. A composition comprising:
means for obtaining at least one natural ingredient;
means for combining the at least one natural ingredient in a container at a predetermined quantity;
means for mixing the at least one natural ingredient;
means for separating components from the at least one natural ingredient; and
means for applying the composition to a surface.

20. A composition consisting of:
at least one ingredient selected from a plurality of ingredients consisting of Frankincense, Myrrh, black seed, and aloe, said plurality of ingredients comprising natural ingredients, said plurality of ingredients further comprising camel hump butter, and/or shea butter, and/or ginseng, and/or a fatty acid, and/or a natural resin, and/or an oil, said composition configured to be applied on a surface, said application comprising rubbing or massaging, said surface comprising skin, said composition further comprising a cream, said Frankincense comprising C₂₀H₃₂O₄, gum, similar to gum arabic 30-36%, 3-acetyl-beta-boswellic acid, alpha-boswellic acid, 4-O-methyl-glucuronic acid, incensole acetate, and phellandrene, said Myrrh comprising a reddish resin, said reddish resin originating from a species of genus Commiphora, said composition further comprising a first formulation, a second formulation, a third formulation, and a fourth formulation, said first formulation comprising said Frankincense, Myrrh, black seed, and aloe, said second composition comprising said Frankincense, Myrrh, camel butter, shea butter, and aloe, said third formulation comprising Frankincense, Myrrh, shea butter, ginseng, and natural resin, said fourth formulation comprises Frankincense, Myrrh, and a natural resin.
